SEQUENCE LISTING (23 sequences)

SEQ ID NO: 1 - protein, 211 aa, Human
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 2 - protein, 211 aa, Mouse
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 3 - protein, 211 aa, Rat
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 4 - protein, 215 aa, Chicken
LWDVVAVCVVLLNTVSTSPLPAGKMPPEGPPSVVEGPEDDLSPISLPPPYAVHSDSNMPEDYPDQFDEVVDFIQATIKRLRRSPDKQTPIFSRRERNRQSAATNVENSSKKGRRNQKGKNRGCVLTAIHLNVTDLDLGYETKEELIFRYCSGSCDAVETTYDKILKNLTRKKKLVNDKVRQACCRPTAFDDDLSFLDDNLVYHILKKHSAKRCGC

SEQ ID NO: 5 - protein, 77 aa, Human
MKLWDVVAVCLVLLHTASAFPLPAGKRPPEAPAEDRSLGRRRAPFALSSDSNMPEDYPDQFDDVMDFIQATIKRLKR

SEQ ID NO: 6 - protein, 77 aa, Mouse
MKLWDVVAVCLVLLHTASAFPLPAGKRLLEAPAEDHSLGHRRVPFALTSDSNMPEDYPDQFDDVMDFIQATIKRLKR

SEQ ID NO: 7 - protein, 77 aa, Rat; identical to SEQ ID NO: 6

SEQ ID NO: 8 - protein, 82 aa, Chicken
LWDVVAVCVVLLNTVSTSPLPAGKMPPEGPPSVVEGPEDDLSPISLPPPYAVHSDSNMPEDYPDQFDEVVDFIQATIKRLRR

SEQ ID NO: 9 - protein, 5 aa, Artificial Sequence
  Human, Rat, Mouse Peptide
FPLPA

SEQ ID NO: 10 - protein, 11 aa, Human
  misc_feature (11)..(11): alpha amidation
PPEAPAEDRSL

SEQ ID NO: 11 - protein, 11 aa, Artificial Sequence
  Mouse, Rat Peptide
LLEAPAEDHSL

SEQ ID NO: 12 - protein, 10 aa, Human
SPDKQMAVLP

SEQ ID NO: 13 - protein, 10 aa, Artificial Sequence
  Mouse, Rat Peptide
SPDKQAAALP

SEQ ID NO: 14 - protein, 10 aa, Chicken
SPDKQTPIFS

SEQ ID NO: 15 - protein, 17 aa, Human
  misc_feature (17)..(17): alpha amidation
ERNRQAAAANPENSRGK

SEQ ID NO: 16 - protein, 17 aa, Artificial Sequence
  Mouse, Rat Peptide
ERNRQAAAASPENSRGK

SEQ ID NO: 17 - protein, 17 aa, Chicken
  misc_feature (17)..(17): alpha amidation
ERNRQSAATNVENSSKK

SEQ ID NO: 18 - protein, 14 aa, Human
  misc_feature (14)..(14): alpha amidation is not certain
YPIKPEAPGEDAFL

SEQ ID NO: 19 - protein, 36 aa, Human
YPIKPEAPGEDASPEELNRYYASLRHYLNLVTRQRY

SEQ ID NO: 20 - protein, 134 aa, Human
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 21 - protein, 134 aa, Mouse
SPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 22 - protein, 134 aa, Rat
SPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 23 - protein, 133 aa, Chicken
SPDKQTPIFSRRERNRQSAATNVENSSKKGRRNQKGKNRGCVLTAIHLNVTDLDLGYETKEELIFRYCSGSCDAVETTYDKILKNLTRKKKLVNDKVRQACCRPTAFDDDLSFLDDNLVYHILKKHSAKRCGC

(19) United States
(12) Patent Application Publication
(10) Pub. No.: US 2006/0258576 A1
(43) Pub. Date: Nov. 16, 2006

(54) Gdnf-related neuropeptides

(75) Inventors: Tiina Immonen, Vantaa (FI); Hannu Sariola, Helsinki (FI); Anniina Alakuijala, Espoo (FI); Michael Pasternack, Vantaa (FI); Christophe Roos, Esbo (FI)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Licentia, Ltd, Helsinki (FI)

(21) Appl. No.: 10/570,460

(22) PCT Filed: Sep. 3, 2004
(86) PCT No.: PCT/IB04/03101

§ 371 Date: Jun. 15, 2006

Related U.S. Application Data
(60) Provisional application No. 60/500,613, filed on Sep. 5, 2003.

(51) Int. Cl. A61K 38/19; C07K 14/475; C07K 16/22; C07H 21/04; C12P 21/06

(52) U.S. Cl. 514/12; 530/399; 435/69.1; 435/320.1; 435/325; 530/388.25; 536/23.5

(57) ABSTRACT

The invention provides novel neuropeptides having amino acid sequences derived from GDNF precursors and homologs thereof, as well as compositions containing the novel neuropeptides. The invention also provides polynucleotides encoding the neuropeptides, antibodies that specifically bind to the neuropeptides, and methods of making and using all of the above, particularly in the treatment of motor disorders, neuropathic pain, and for modulating excitatory neurotransmission.

### CROSS-REFERENCE TO RELATED APPLICATIONS

This application claims benefit of U.S. Provisional Patent Application No. 60/500,613, filed Sep. 5, 2003, the disclosure of which is hereby incorporated by reference herein in its entirety.

### FIELD OF THE INVENTION

The present invention is directed to neuropeptides derived from the precursor of glial cell line-derived neurotrophic factor (GDNF) and those of GDNF homologs, and use of these novel neuropeptides.

### BACKGROUND OF THE INVENTION

Neuropeptides are polypeptides that serve effector functions in the nervous system. Most vertebrate polypeptide hormones and neuropeptides of the constitutive secretory pathway are synthesized as large biologically inactive proproteins and are activated through endoproteolytic cleavage of the propeptide. The enzymes responsible for the activation: furin, PACE4, PC4, PC5/6 and PC7/8, are members of the prohormone convertase (PC) family. Known substrates include pro-β-nerve growth factor (Bresnahan P. et al. (1990) J. Cell Biol. 111:2851-2859), proinsulin-like growth factor-IA (Duguay S. et al. (1997) J. Biol. Chem. 272(10): 6663-6670) and bone morphogenic protein-4 (BMP-4) (Cui Y. et al. (1998) EMBO J. 17(16): 4735-4743; Constam D. and E. Robertson (1999) J. Cell Biol. 144(1): 139-149).

GDNF is a protein that is initially synthesized as a larger proprotein (precursor) and is subsequently proteolytically processed to form mature GDNF. GDNF belongs to the transforming growth factor β (TGFβ)-family of proteins, which are characterized by a “cysteine knot”: a pattern of intertwined loops consisting of three intramolecular disulfide bonds. In addition, one of the cysteines conserved within the family is used for homodimer formation. These bonds are formed already in the endoplasmic reticulum after translocation and signal sequence removal, whereas furin has been localized in trans-Golgi (Shapiro J. et al. (1997) J. Histochem. Cytochem. 45(1): 3-12). The cysteine-bonding pattern of substrates, such as GDNF precursor, affects the accessibility of the protein to processing.

GDNF signals via its receptor cRet, a receptor tyrosine kinase, and the co-receptor GDNF family receptor α1 (GFRα1). Other co-receptors act in conjunction with cRet to transduce a signal for other ligands. For instance, cRet and the co-receptor GFRα2 are required for neurturin signalling, cRet and its co-receptor GFRα3 are required for artemin signalling, and cRet and GFRα4 are required for persephin signalling. Evolutionary studies indicate that GDNF could represent an ancestral form of neurotrophic factors.

In addition to neuronal tissue, GDNF has been found to be expressed in a variety of non-neuronal tissues of animals including developing skin, whisker pad, kidney, stomach, testis, developing skeletal muscle, ovary, lung, and adrenal gland (Trupp, M. et al. (1995) J. Cell Biol. 130(1): 137-48). GDNF has been found to play a role in nephrogenesis (Moore, M. W. et al. (1996) Nature 382(6586):76-9); apoptosis-driven hair follicle involution (Botchkareva, N. V. et al. Am. J. Pathol. 156(3):1041-1053); heart development (Hiltunen, J. O. (2000) Dev Dyn 219(1):28-39); and maintenance of the adult enteric nervous system (Peters, R. J. et al. (1998) J. Auton Nerv. Syst. 70(1-2): 115-22). Both the rodent and human GDNF precursors contain several putative target sites for the successive actions of PCs and the enzymes required for C-terminal amidation, carboxypeptidase E (CPE) and peptidylglycine a-amidating monoxygenase (PAM) (FIG. 1 A). Areas and timing of expression of these enzymes overlap also the sites of GDNF synthesis (Shafer et al. (1993) J. Neurosci. 13(3): 1258-1279, Zheng et al. (1997) Dev. Biol. 181(2): 268-283, Zhang et al. (1997) Dev. Biol. 192(2): 375-392). Cleavage of proGDNF at the PC substrate sites could result in the formation of several amidated peptides (FIG. 1, Table 1).

The polypeptides of the invention have effects in the nervous system. In addition, the polypeptides of the invention may have effects outside of the nervous system as well. Such activities are also embraced by the invention. It is known for some neuropeptides that the neuropeptides have other effects apart from the nervous system. For example, they regulate the secretory functions of adrenal cortex and pancreas (Renshaw, D. et al (2000) Endocrinol. 141(1): 169-73 and act as immunomodulators (Krantic S. (2000) Peptides 21(12):1941-64; Wiedermann C. J. (2000) Peptides 21(8):1289-98). Therefore, the invention embraces effects of the neuropeptides acting in the nervous system and apart from the nervous system.

Interaction of GDNF with its receptors cRet and GFRα1 has been suggested to have a role in a variety of diseases and disorders including: neuropathic pain (Boucher, T. J. et al. (2000) Science 290(5489): 124-7); motor diseases such as amyotrophic lateral sclerosis (ALS) and X-linked spinal and bulbar muscular atrophy (SBMA) (Yamamoto, M. et al. (1999) Neurochem. Res. 24(6): 785-90); neurodegenerative diseases such as Parkinson's disease (Walton, K. M. (1999) Mol. Neurobiol. 19(1): 43-59); renal diseases (Onodera, H. et al. (1999) Nephrol. Dial. Transplant. 14(6): 1604-5); and epileptic syndromes (Kokaia, Z. et al. (1999) Eur. J. Neurosci. 11(4): 1202-16).

There is a need in the art to further study GDNF, and to develop effective treatments for motor neuropathies, neurodegenerative diseases, renal disorders and neuropathic pain based on the interaction of GDNF and homologs thereof with their receptors.

### SUMMARY OF THE INVENTION

The present invention provides isolated neuropeptides derived from the GDNF precursor and its homologs. Surprisingly, polypeptides of the invention have an effect on hippocampal neurones that mimics or is opposite to the effect of neuropeptide Y (NPY) and polypeptide YY (PYY). The GDNF precursor and homologs may be derived from animal GDNFs, including, but not limited to mammalian and vertebrate GDNFs. Examples of GDNF precursor and homologs include, but are not limited to precursors of human GDNF (SEQ ID NO:1), mouse GDNF (SEQ ID NO:2), rat GDNF (SEQ ID NO:3), and chicken GDNF (SEQ ID NO:4). The invention also provides isolated neuropeptides derived from human GDNF precursor comprising the amino acid sequences of SEQ ID NO:9, SEQ ID NO:10, SEQ ID NO:12, and SEQ ID NO:15.

The invention further provides isolated neuropeptides derived from the mouse GDNF precursor protein comprising the amino acid sequence of SEQ ID NO:9, SEQ ID NO:11, SEQ ID NO:13, and SEQ ID NO:16, as well as isolated neuropeptides derived from the rat GDNF precursor protein comprising the amino acid sequence of SEQ ID NO:9, SEQ ID NO:11, SEQ ID NO:13, and SEQ ID NO:16.

The invention also provides isolated neuropeptides derived from the chicken GDNF precursor protein comprising the amino acid sequence of SEQ ID NO:14 and SEQ ID NO:17.

Alpha-amidated neuropeptides are also provided. In specific embodiments, the alpha-amidated neuropeptides have the amino acid sequence of SEQ ID NO:9, SEQ ID NO:10, SEQ ID NO:11, SEQ ID NO:15, and SEQ ID NO:16, or SEQ ID NO:17.

The invention provides polynucleotide sequences encoding the various neuropeptides of the invention. Plasmids comprising polynucleotides encoding the isolated neuropeptides of the invention are also embraced by the invention. In certain embodiments, the plasmids are expression vectors for expressing the polynucleotides encoding the neuropeptides of the invention.

The invention also provides transformed cells in which a prokaryotic or eukaryotic cell is transfected with a plasmid comprising the polynucleotides encoding the neuropeptides of the invention. The cells can be of vertebrate, invertebrate, bacterial or yeast origin. In preferred embodiments, the cells are of vertebrate, specifically mammalian, origin.

The invention provides methods for producing the neuropeptides of the invention such as through recombinant molecular technology, chemical synthesis, and purification from natural sources. The peptides may also be cleaved in vitro after first isolating larger precursors from cells.

The invention also provides methods of modulating neuronal responses by administering to an animal the isolated polypeptides derived from GDNF precursor. In yet a further aspect, the present invention relates to a method for modulating neuronal responses in mammals by administering to an animal the isolated polypeptides derived from the GDNF precursor.

The invention also provides methods of modulating responses in non-neuronal tissues both in vivo and in vitro. In preferred embodiments the non-neuronal tissues include, but are not limited to renal tissue, testicular tissue, skin, cardiac tissue, gastrointestinal tissue, skeletal muscle, ovarian tissue, lung tissue, and adrenal tissue.

In yet another aspect, the present invention relates to a probe for identifying GDNF neuropeptide receptors in vertebrates. The probe comprises a polypeptide derived from GDNF or a GDNF homolog. In specific embodiments, the probes have the amino acid sequence of SEQ ID NOs: 9-17.

The invention also provides methods of modulating excitatory neuronal transmission by administering to a subject an effective amount of at least one neuropeptide of the invention. In some embodiments, the invention provides methods of modulating excitatory neuronal transmission by administering to a subject an effective amount of at least one neuropeptide of the invention.

The invention further provides methods of modulating cRet by administering compositions that contain at least one neuropeptide of the invention.

The invention further provides methods of modulating NPY—Yᵢ receptors by administering compositions that contain at least one neuropeptide of the invention.

The invention also provides compositions and methods for treating motor disorders such as ALS; neurodegenerative disorders such as Parkinson's Disease; epileptic syndromes; renal disorders; skin disorders; testicular disorders; and neuropathic pain. The compositions contain at least one GDNF precursor derived neuropeptide, homolog, or fusion protein thereof, or at least a portion of an anti-neuropeptide antibody that specifically binds to a GDNF precursor derived neuropeptide or homolog thereof.

### BRIEF DESCRIPTION OF THE DRAWINGS

The application file contains at least one drawing executed in color. Copies of this patent application publication with color drawings will be provided by the Office upon request and payment of the necessary fee.

FIG. 1. An amino acid sequence alignment of the precursors of human GDNF (SEQ ID NO:1), mouse GDNF (SEQ ID NO:2), rat GDNF (SEQ ID NO:3) and chicken GDNF (SEQ ID NO:4). The proregions of each GDNF precursor are shown overlined with an arrow and include: human (SEQ ID NO:5), mouse (SEQ ID NO:6), rat (SEQ ID NO:7) and chicken (SEQ ID NO:8), GDNF proregion. The cleavage sites are C-terminal to the sites marked in bold letters. Cysteine residues are indicated with boxes, and the mature GDNF sequence for human, mouse, rat, and chicken (SEQ ID:s 20-23, respectively) is doubly overlined. Putative prohormone convertase (PC) processing sites in human, mouse, rat and chicken GDNF are shown in boldface letters.

FIG. 2. Amino acid sequence alignment of hPEP2 (SEQ ID NO:10), rPEP2 (SEQ ID NO:11), hPYY (SEQ ID NO:19), and hPYY2 (SEQ ID NO:18).

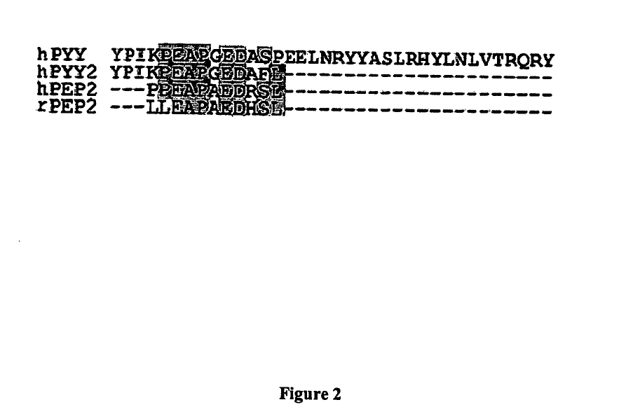

FIG. 3. Binding of iodinated peptides SEQ ID NO:9 and SEQ ID NO:11 to rodent tissues. The I¹²⁵-labelled SEQ ID NO:11 displayed intensive binding to slices of adult rat brain (A-B), in contrast to I¹²⁵-labelled SEQ ID NO:10 (C-D). The bright (A, C) and dark field (B, D) images of sections containing the hippocampal area from the peptide-incubated brain tissue are shown. I¹²⁵-labelled SEQ ID NO:9 (E, H) bound to embryonic (E15) mouse tissues. In the gut (E, F) and the metanephric kidney capsule (G, H), the binding of I¹²⁵-labelled SEQ ID NO:9 (E, G) and the immunohistochemical staining with neuron-specific Tuj1 antibody (F, H) have similar distributions. In testis, I¹²⁵-labelled SEQ ID NO:9 binding to the interstitium (I) was complementary both to the expression of GDNF and to the localization of neurons with Tuj1 within the seminiferous tubules (J).

FIG. 4. FIG. 4. SEQ ID NO:11 (BEP) increases synaptic transmission via a NEM (N-ethylmaleimide)-sensitive receptor in the adult rat hippocampus. Examples of individual recordings of the effect of SEQ ID NO:11 on the stimulus-induced population spikes (pSpikes) and the population excitatory postsynaptic potentials (pEPSPs) in the absence (A) or the presence (B) of NEM are shown. The increase in presynaptic volley is seen only in the absence of NEM (A). Empty arrowheads indicate stimulation. Summaries of the measurements of pSpikes (C) and pEPSPs (D) are shown as fractions of control (control=1). SEQ ID NO:11, but not SEQ ID NO:9 (NRP) or SEQ ID NO:15 (hPEP4), substantially increases both pSpikes and pEPSPs. NEM alone also increases both pSpikes and pEPSPs, but no further increase is detected when also SEQ ID NO:11 is added. In (C) and (D) the effect of SEQ ID NO:11 is compared to the baseline at the beginning of an experiment. 10 nM SEQ ID NO:11, 20 nM SEQ ID NO:9 and SEQ ID NO:15, and 250 μM NEM was used. ns=not significant.

FIG. 5. Binding of the control peptide SEQ ID NO:15 to embryonic mouse testis (A) and gut (B).

FIG. 6. Effects of increasing SEQ ID NO:11 concentrations on pEPSP and pSpike. 10 nM SEQ ID NO:11 was chosen for further experiments.

### DETAILED DESCRIPTION OF ILLUSTRATIVE EMBODIMENTS

The practice of the present invention, unless otherwise indicated, employs conventional methods of molecular biology and recombinant DNA techniques known to those of ordinary skill of the art. Such techniques are known and fully explained in the literature, and are available in such reference texts as Sambrook, et al., MOLECULAR CLONING: A LABORATORY MANUAL (2nd Edition, 1989); DNA CLONING: A PRACTICAL APPROACH, Vols. I & II (D. Glover, ed.); METHODS IN ENZYMOLOGY (S. Colowick and N. Kaplan eds., Academic Press, Inc.); HANDBOOK OF EXPERIMENTAL IMMUNOLOGY, Vols. I-IV (D. M. Weir and C. C. Blackwell Eds., Blackwell Scientific Publications); and FUNDAMENTAL VIROLOGY, 2nd Edition, Vols. I & II (B. N. Fields and D. M. Knipe, Eds.). The reference works, patents, patent applications, and scientific literature, including accession numbers to GenBank database sequences, referred to herein are hereby incorporated by reference in their entirety. Any conflict between any reference cited herein and the specific teachings of this specification shall be resolved in favor of the latter.

1. Definitions

Various definitions are made throughout this document. Unless otherwise indicated, the words have the meaning that would be attributed to those words by one skilled in the art. Words specifically defined either below or elsewhere in this document have the meaning provided in the context of the present invention as a whole and as are typically understood by those skilled in the art. Any conflict between an art-understood definition of a word or phrase and a definition of the word or phrase as specifically taught in this specification shall be resolved in favor of the latter. Headings, however, are provided herein for convenience, and are not to be construed as limiting in any way.

As used herein, the terms “a,” “an,” and “the” refer to the singular and the plural.

As used herein “GDNF precursor” refers to a glial cell line derived neurotrophic factor propeptide that contains the N-terminal prosequence of GDNF joined to the N-terminal sequence of the mature GDNF. The GDNF precursor may also include the signal sequence.

As used herein, “mature GDNF” refers to glial cell line-derived neurotrophic factor of vertebrates that has been cleaved from the N-terminal prosequence of the GDNF precursor. The vertebrates may be mammals or non-mammalian vertebrates. In preferred embodiments, the mature GDNF is derived from mammals, particularly rodents, non-human primates and humans.

As used herein “GDNF prosequence” refers to the N-terminal portion of the GDNF precursor that is cleaved from the GDNF precursor upon formation of mature GDNF.

As used herein “proteolytically processed fragment of a GDNF precursor” refers to a fragment of the GDNF precursor formed by proteolytic cleavage of the GDNF precursor.

As used herein, “homologs” refers to analogous sequences in other organisms that have substantially the same amino acid sequence of the reference sequence and perform at least one analogous function as the reference sequence. For example, a homolog of GDNF precursor is a polypeptide derived from any animal that has substantial identity at the amino acid level as human, mouse, rat, or chicken peptides.

As used herein, the term “complementary” refers to a nucleic acid sequence, which is able to associate with another nucleic acid according to the rules of Watson-Crick base-pairing. For example, an mRNA is complementary to a DNA strand of the gene, which encodes it; a DNA sequence and its complementary DNA sequences can form a hydrogen-bonded duplex.

As used herein, the term “polynucleotide” refers to a chain of nucleotides linked by phosphodiester bonds, including modifications thereof for improving stability, and includes RNA and DNA.

The term “nucleic acids” as used herein includes, for example, genomic DNA, mRNA, and cDNA.

As used herein, the term “polypeptide” refers to a chain of amino acid residues linked by peptide bonds.

Any claims to sequences herein encompass those insubstantial alterations that can be made to a sequence without effecting function, i.e., substantially the same sequences. For example, a change in a nucleotide within a codon that results in the same amino acid as originally encoded by the codon is “substantially the same sequence.” Also, a conservative amino acid substitution within the sequence that does not affect function is also “substantially the same sequence.”

The term “bind” as used herein refers to the interaction between GDNF or GDNF precursor-derived peptides and their receptors or binding proteins, the binding being of a sufficient strength and for a sufficient time to allow the detection of said binding under the conditions of the assays disclosed herein.

The term “about” in reference to a numerical value means 10% of the numerical value, more preferably 5%, most preferably 2%.

The term “probe” in relation to a polynucleotide or polypeptide includes a probe of sufficient length to signify specific binding. The polynucleotides can also be utilized for gene therapy, using both in vivo and ex vivo techniques. The polypeptides can also be used therapeutically, and to prepare monoclonal and polyclonal antibodies using known techniques.

The term “effect” means an alteration or change. An effect can be positive, such as causing an increase in some material, or negative, e.g., antagonistic or inhibiting.

When referring to a polypeptide, examples of isolated polypeptides include, but are not limited to precursor polypeptides, partially or substantially purified polypeptides, peptides produced in a heterologous cell, and synthetic peptides. Preferably the isolated polypeptides of the invention are free of additional amino acids in the naturally occurring protein and may be substantially purified from culture medium or chemical precursors when chemically synthesized.

As used herein, “synthesized” refers to polynucleotides and polypeptides produced by purely chemical, as opposed to enzymatic, methods. The synthesis may be complete or partial synthesis.

As used herein, “activity” refers to any measurable indicia suggesting or revealing binding, either direct or indirect; affecting a response, i.e. having a measurable affect in response to some exposure or stimulus, including, for example, the affinity of a compound for directly binding a polypeptide or polynucleotide of the invention, or, for example, measurement of amounts of upstream or downstream proteins or other similar functions after some stimulus or event.

As used herein, a “neuropeptide” is a polypeptide possessing at least one neuropeptide activity, and may also have a function or functions outside of the nervous system.

As used herein “neuropeptide activity” refers to an activity linked to both neural cells and non-neural cells and includes, but is not limited to such activities as (a) receptor binding (e.g., to the cRet or NPY—Yᵢ receptors), (b) stimulation of neural cells to produce an effect; (c) stimulation of tyrosine kinase activity (d) the ability to modulate the excitatory synaptic input, for example, into primary cells in the CA1 area of hippocampal brain slices; (e) immunogenicity that elicits a neuropeptide-specific immune response in an animal; (f) stimulation of renal tubule regeneration, cessation of hair loss or stimulation of hair growth (g) stimulation or inhibition of spermatogenesis (h) stimulation of a GDNF-related function in ovarian tissue, lung tissue, gastrointestinal tissue, skeletal muscle, cardiac tissue and/or adrenal tissue, and/or (h) immunogenicity that elicits an immune response in an animal that cross-reacts with GDNF precursor or a homolog thereof. Polypeptides that exhibit neuropeptide activity include, for example, the neuropeptides of SEQ ID NOs: 9-17, and 5-8. Thus, the term “neuropeptide activity” encompasses functions within the nervous system as well as functions outside of the nervous system.

As used herein “motor disorders” refers to muscle dysfunction, preferably neural-mediated muscle dysfunction. Non-limiting examples of motor disorders include such neuropathies as amyotrophic lateral sclerosis (ALS), X-linked spinal and bulbar muscular atrophy (SBMA), chronic inflammatory demyelinating polyneuropathy, and demyelinating Guillain-Barre syndrome.

As used herein “neurodegenerative disorders” refers to disorders characterized by progressive loss of neural function. A non-limiting example includes Parkinson's disease.

As used herein “neuropathic pain” refers to chronic pain that persists after the initial insult to the body has healed. Neuropathic pain may be marked by a variety of symptoms, including, but not limited to hyperalgesia, and hyperaesthesia.

As used herein “epileptic syndromes” refers to diseases characterized by electrophysiological disorders of the brain. Non-limiting examples include epilepsy and epileptic seizures.

As used herein “renal disorders” refers to chronic disorders of the kidney and ureters marked by decline in kidney function over time as well as to acute damage to the kidneys resulting in loss of renal function.

As used herein “testicular disorders” refers to disorders of the testis resulting in reduced spermatogenesis.

As used herein “skin disorder” refers to a disorder of the dermal layers that results in hair loss.

As used herein “cardiac disorders” refers to a disorder of the cardiac tissue resulting in abnormal heart rate or rhythm.

As used herein a “pharmaceutically acceptable carrier” refers to a compound or mixture of compounds that are suitable excipients for the delivery of drugs, antibodies, polynucleotides or polypeptides.

Unless indicated otherwise, as used herein, the abbreviations in lower case (gdnf) refer to a gene, cDNA, RNA or nucleic acid sequence, while the upper case versions (GDNF) refers to a protein, polypeptide, peptide, oligopeptide, or amino acid sequence. Full-length genomic DNA for human gdnf and cDNA (with excised introns) as well as other nucleic acid molecules encoding a GDNF without the neuropeptide region are encompassed by “gdnf.”

As used herein, the term “antibody” is meant to refer to complete, intact antibodies, and Fab, Fab′, F(ab)₂, and other fragments thereof. Complete, intact antibodies include monoclonal antibodies such as murine monoclonal antibodies, chimeric antibodies, anti-idiotypic antibodies, anti-anti-idiotypic antibodies, and humanized antibodies.

2. Polypeptides

The neuropeptides of the invention may be derived from human GDNF and GDNF homologs, including, but not limited to mouse, rat and chicken GDNF. The neuropeptides of the invention may be GDNF precursors having at least one neuropeptide activity. In addition, the polypeptides may also have activity outside of the nervous system. The neuropeptides of the invention may also be proteolytically processed fragments of GDNF precursors and homologs that possess at least one neuropeptide activity. As used herein, “proteolytically processed” refers to naturally occurring fragments from endogenous enzymatic digestion of the GDNF protein or homolog, or artificially induced digestion of the GDNF precursor protein or homolog in vitro.

The proteolytic fragments of the invention comprise amino acid sequences of GDNF precursor and homologs and homologs that are the result of enzymatic digestion, containing a protease cleavage site and/or which have at least one neuropeptide activity. In preferred embodiments, the proteolytic fragments of the invention comprise the neuropeptides of SEQ ID NOs: 9-17 and 5-8 (derived from GDNF).

The polypeptides of the invention may be naturally processed neuropeptides that are isolated from cells. Alternatively, the GDNF precursor protein may be isolated from cells and processed in vitro to obtain the neuropeptides. Alternatively, the neuropeptides of the invention may be produced using recombinant molecular technology, by directly producing the peptides, or by producing larger precursors (including the entire GDNF precursor) and subsequently processing the protein to produce the neuropeptides. Another alternative is to produce the neuropeptides of the invention chemically.

The polypeptides of the invention include polypeptide sequences that have at least about 99%, at least about 95%, at least about 90%, at least about 85%, at least about 80%, at least about 75%, at least about 70%, at least about 65%, at least about 60%, at least about 55%, at least about 50%, or at least about 45% identity and/or homology to the preferred polypeptides of the invention, the GDNF precursor-derived neuropeptides or homologs thereof. The preferred polypeptides of the invention contain the amino acid sequences of SEQ ID NOs: 9-17 and 5-8. In addition, the polypeptides of the invention include polypeptides that are modified. The neuropeptides of the invention may be glycosylated, lipidated, and/or phosphorylated. Furthermore, chemical modification may include any chemical modification or functional group substitution that allows the polypeptide to retain at least one neuropeptide activity (such as, but not limited to the ability to elicit an immune response against unmodified neuropeptide), or at least one activity outside of the nervous system. In a preferred embodiment, the chemical modification is α-amidation. Preferred examples of the α-amidated neuropeptides of the invention include α-amidated peptides having the amino acid sequence of SEQ ID NOs: 9, 10, 11, 15, 16, and 17.

The neuropeptides of the invention may also include additions, substitutions or deletions as necessary or helpful in using the neuropeptides of the invention as described herein. For example, neuropeptides may be engineered with an additional N-terminal methionine residue to aid in recombinant expression of the neuropeptide. Conservative substitutions may also be made in the amino acid sequence of the neuropeptide provided at least one neuropeptide function is retained by the modified peptide (such as but not limited to the ability to elicit an immune response against native neuropeptide), or at least one activity outside of the nervous system is retained by the modified peptide. Conservative substitutions are well known in the art. References for determining conservative substitutions include from WO 97/09433 published Mar. 13, 1997, and Lehninger, BIOCHEMISTRY, Second Edition, Worth Publishers, Inc. NY, N.Y. (1975), pp. 71-77, the disclosures of which are incorporated herein by reference.

Amino acid insertions, deletions and/or substitutions may be introduced into the neuropeptides of the invention by standard techniques, such as site-directed mutagenesis and PCR-mediated mutagenesis. Alternatively, the neuropeptides may be chemically synthesized as desired. Preferably, conservative amino acid substitutions are made at one or more amino acid positions, which are determined to be non-essential, i.e., amino acid residues which may be substituted without substantial effect on biological activity.

3. Polynucleotides

The neuropeptides of the invention may be encoded by any polynucleotide sequence that results in the amino acid sequences of SEQ ID NOs: 9-17, and 5-8 or homologs thereof which contain a protease cleavage site and/or at least one neuropeptide activity. The polynucleotides of the invention may encode vertebrate GDNF precursor (e.g., human GDNF precursor (SEQ ID NO:1), mouse GDNF precursor (SEQ ID NO:2), rat GDNF precursor (SEQ ID NO:3), and chicken GDNF precursor (SEQ ID NO:4) that is subsequently proteolytically processed into the neuropeptides of the invention. Due to the degeneracy of the genetic code, a multitude of nucleic acid sequences may encode the neuropeptides of the invention. The polynucleotides of the invention include all polynucleotides that encode neuropeptides having a polypeptide sequence derived from a GDNF precursor or homolog thereof.

The polynucleotides encoding the neuropeptides of the invention may be isolated polynucleotide sequences or fragments thereof, sequences having complementarity to the isolated polynucleotide sequences or fragments thereof, or may be part of larger molecules, such as plasmids, for example. However, the polynucleotides of the invention exclude intact polynucleotide sequences in their native state as in the chromosome or the genome of the organism from which it is derived.

One aspect of the present invention is directed to vectors, or recombinant expression vectors, comprising any of the nucleic acid molecules encoding the neuropeptides of the invention. Vectors are used herein either to amplify DNA or RNA encoding the neuropeptides and/or to express DNA which encodes the neuropeptides. As used herein, the terms “vector” and “plasmid” are used synonymously. These refer to nucleic acid molecules capable of transporting another nucleic acid to which it has been linked. Generally, vectors are capable of autonomous replication in a host cell into which they are introduced. Other vectors such as non-episomal mammalian vectors integrate into the genome of a host cell upon introduction. These replicate as part of the host genome. Expression vectors contain nucleic acid sequences that direct the expression of polynucleotide sequences that are operatively linked to the functional sequences of the expression vector. The invention also embraces polynucleotides cloned in viral vectors, including, but not limited to replication defective retroviruses, adenoviruses and adeno-associated viruses. The expression vectors may also include polynucleotide sequences that encode a selectable marker (such as a gene that confers drug-resistance), and other sequences to allow formation of fusion proteins. Typical drug resistance genes allow resistance to tetracyline, kanamycin, and ampicillin. In some embodiments, the fusion protein aids in protein purification, solubility, or increased production. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione-S-transferase (GST), maltose E binding protein, or protein A, respectively, to the target recombinant protein. Non-limiting examples of suitable inducible non-fusion E. coli expression vectors include pTrc (Amrann et al., (1988) Gene 69:301-315) and pET 1 d (Studier et al., GENE EXPRESSION TECHNOLOGY: METHODS IN ENZYMOLOGY 185, Academic Press, San Diego, Calif. (1990) 60-89).

Expression of proteins may be performed in prokaryotic and eukaryotic cells using appropriate vectors that contain functional sequences that are functional in the particular cell type. “Functional sequences” refers to polynucleotide sequences that direct a function of gene expression. Non-limiting examples of functional sequences include promoters, enhancers, termination sequences, and polyadenylation sequences.

In another embodiment, the neuropeptides are cloned into eukaryotic expression vectors for expression in suitable eukaryotic cells. Examples of vectors for expression in yeast S. cerivisae include pYepSec1 (Baldari, et al., (1987) EMBO J6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.). Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39). Examples of mammalian expression vectors include pCDM8 (Seed (1987) Nature 329:840) and pMT2PC (Kaufman et al. (1987) EMBO J. 6: 187-195).

In some embodiments, the recombinant mammalian expression vector is capable of directing expression of the neuropeptides in a particular cell type. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banedi et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev. 3:537-546).

4. Suitable Host Cells

Suitable host cells for expression of the neuropeptides of the invention include, but are not limited to, prokaryotes, and eukaryotes. Prokaryotic expression vectors are used in conjunction with suitable prokaryotic host cells. The type of prokaryotic cell used is not particularly limited, but preferred examples include bacteria of the genera Escherichia, Bacillus, Salmonella, Pseudomonas, Streptomyces, and Staphylococcus.

Eukaryotic expression vectors are used in conjunction with eukaryotic cells. Preferred host cells include, but are not limited to, yeast cells, insect cells, HeLa cells, Chinese hamster ovary cells (CHO cells), African green monkey kidney cells (COS cells), human 293 cells, and murine 3T3 fibroblasts. Preferred eukaryotic host cells include, but are not limited to, the genera Saccharomyces, Pichia, Kluveromyces; Spodoptera frugiperda cells, Drosophila Schneider cells, HeLa cells, CHO cells, COS cells, 293 cells and 3T3 cells.

5. Antibodies

The antibodies of the invention include antibodies that specifically bind the neuropeptides of the invention. As such, the origin and isotype of the antibodies is not particularly limited. The antibodies may be raised in any antibody-producing animal and may be of any isotype, including, but not limited to IgA, IgD, IgM, IgG, IgE, monoclonal antibodies, chimeric antibodies, grafted antibodies, humanized antibodies, superhumanized antibodies, provided the antibodies specifically bind to at least one neuropeptide of the invention. The antibodies of the invention also include fragments of antibodies that specifically bind at least one neuropeptide of the invention, single chain antibodies, and anti-idiotypic antibodies generated by immunizing an animal with any of the specific anti-neuropeptide antibodies of the invention.

The term “monoclonal antibody” as used herein, refers to an immunoglobulin molecule that contains a single species of an antigen binding that has affinity for a particular epitope (of a given neuropeptide of the invention).

The anti-idiotypic antibodies include, but are not limited to internal image anti-idiotypic antibodies. The antibodies and anti-idiotypic antibodies of the invention may be generated by methods which are well known in the art, and which may be found in a variety of references, such as the HANDBOOK OF EXPERIMENTAL IMMUNOLOGY, Vols. I-IV (D. M. Weir and C. C. Blackwell eds., Blackwell Scientific Publications).

Methods of immunizing animals and generating antibodies, monoclonal antibodies and anti-idiotypic antibodies are well established and known to those of ordinary skill in the art. U.S. Pat. No. 4,946,778 describes a method for the production of single chain antibodies, and is incorporated herein by reference. Non-human antibodies can be “humanized” by techniques well known in the art (e.g., U.S. Pat. No. 5,225,539). Grafted antibodies are those that are humanized by grafting non-human CDRs onto a human antibody constant region, or grafting the non-human CDRs onto a consensus antibody framework sequence. Further changes can then be introduced into the antibody framework (such as those derived from the same organism as the origin of the CDR) to modulate affinity or immunogenicity (“superhumanized” antibodies).

Internal image anti-idiotypic antibodies are raised against antibodies against the neuropeptide that mimic the neuropeptide. These antibodies and methods of preparing them are described in Bona and Kohler (1984) ANTI-IDIOTYPIC ANTIBODIES AND INTERNAL IMAGE, IN MONOCLONAL AND ANTI-IDIOTYPIC ANTIBODIES: PROBES FOR RECEPTOR STRUCTURE AND FUNCTION, Venter J. C., Frasser, C. M., Lindstrom, J. (Eds.) NY, Alan R. Liss, pp 141-149, 1984, which is incorporated herein by reference.

The antibodies of the invention are useful for detecting the neuropeptides of the invention in situ, in assays such as radioimmunoassays (RIA), enzyme-linked immunosorbent assays (ELISA), Western blots, and other well known assays utilizing specific antibodies. The antibodies of the invention are also useful as therapeutics. Such antibodies may be administered to an animal to modulate the effects of a given neuropeptide. Modulating the effects of neuropeptides may include positively or negatively influencing the effect of the neuropeptide. For example, an internal image anti-idiotypic antibody may be administered to increase the effect of the neuropeptide by mimicking the neuropeptide. Antibodies directed against the neuropeptide may act to inhibit the effect of the neuropeptide.

Other antibodies of the invention include portions of antibodies that specifically bind the neuropeptides of the invention and which may be fused to other proteins, or portions thereof.

6. Methods of Identifying Receptors

The neuropeptides of the invention may be used as probes to identify the receptors of the neuropeptides. To identify the neuropeptide receptors, a polypeptide comprising at least a portion of the neuropeptides of the invention (e.g., SEQ ID NOs: 9-17, 5-8) can be used as a probe in screening neuronal and non-neuronal cell lines using techniques well known in the art. As a non-limiting example, binding assays can be performed as follows. Cells can be incubated with a labeled neuropeptide, such as an iodinated neuropeptide, in Dulbecco's phosphate buffered saline and 2 mg/ml bovine serum albumin (BSA) on Millipore Hydrophilic Durapore 96-well filtration plates. Following two hours of vigorous shaking at 4° C., the cells are washed twice with ice-cold binding buffer under a vacuum. Dried filters are liberated and bound ¹²⁵I-neuropeptide is quantified in a gamma counter. Non-specific binding is determined by addition of 500-fold excess of cold ligand to the binding mixtures.

For affinity labelling, labeled neuropeptides (e.g., iodinated neuropeptides) are bound to monolayer cultures of primary neurons or cell lines. Prior to binding, cells are cultured for 48 hours in the presence of NGF on polyornithine/laminin coated dishes. Plated cells are incubated with 10 ng/ml ¹²⁵I-neuropeptide at 4° C. in binding buffer as described above. Ligand/receptor complexes are chemically cross-linked for thirty minutes at room temperature using either disuccinimidyl suberate (DSS) or 1-Ethyl-3(−3-dimethylaminopropyl)-carbodiimide hydrochloride (EDAC) (Pierce Chemical, Rockland, Ill.). Following quenching of the cross-linking reactions, cells are washed twice with 10 mM Tris/HCI buffered saline, 2 mM EDTA, 10% glycerol, 1% NP-40, 1% Triton X-100, 10 μg/ml leupeptin, 10 μg/ml antipain, 50 μg/ml aprotinin, 100 μg/ml benzamidine hydrochloride, 10 μg/ml pepstatin and 1 mM PMSF (proteinase inhibitors from Sigma). Cleared lysates are boiled for 5 min in SDS/β-mercaptoethanol buffer, fractionated by SDS/PAGE on 4-20% gradient electrophoresis gels, and visualized by autoradiography. Molecular weights for the receptors can be obtained by subtracting the weight of the neuropeptide from the estimated molecular weights of cross-linked complexes visualized by SDS/PAGE.

For affinity measurements of cross-linked complexes, cells are incubated on plates as above in the presence of increasing amounts of unlabelled neuropeptide. The samples are fractionated by gradient SDS/PAGE, gels are then dried and specific bands excised according to molecular weights determined from autoradiograms, and counted in a gamma counter.

Other methods of identifying receptors include affinity chromatography using immobilized neuropeptides. Once a receptor candidate is identified, the protein can be subjected to amino acid sequencing or partial amino acid sequencing using standard procedures such as Edmund degradation, and degenerate oligonucleotide probes may be synthesized that correspond to the possible nucleic acid sequence encoding this region of the receptor candidate, and the oligonucleotides may be used as probes to screen cDNA libraries, or may be used as primers in a polymerase chain reaction (PCR) to amplify the appropriate portion of the genomic sequence of the receptor candidate. DNA sequencing may be employed to determine the molecular sequence of the receptor candidate.

Other methods of screening for receptors or modifications of the above methods, involving routine experimentation, would be recognized by one of ordinary skill in the art. Modifications of the methods presented herein may be made without departing from the spirit and scope of the invention.

Without being bound by any particular theory of operation, it is believed that the GDNF precursor derived neuropeptides of the invention interact with the receptor for GDNF (cRet and/or GDNF family receptor α1). By binding to cRet, the GDNF precursor-derived neuropeptides of the invention may stimulate these tyrosine kinase receptors to induce biological responses similar to that induced by the binding of GDNF or a homolog of GDNF. The neuropeptides may, therefore, be used as substitutes for GDNF in therapeutic contexts, as probes, and in assays. Therefore, the neuropeptides of the invention may be used in any utility known for GDNF in which GDNF interacts with one of its receptors. Specific examples of such utilities are described in more detail herein, but include use as therapeutics in neuronal disorders and non-neuronal disorders, including, but not limited to renal disorders, cardiac disorders, skin disorders, and testicular disorders.

The peptides of the invention may also interact with other known receptors. For example, and not by way of limitation, two of the neuropeptides of the invention (SEQ ID NO:9 and SEQ ID NO:11) modulate the excitatory synaptic input into primary cells in the rat CA1 as shown by acute rat hippocampal brain slice preparation studies (see Example 5). Furthermore, the homology between the peptides (shown in Example 2) SEQ ID NO:10, SEQ ID NO:11 and Peptide YY (PYY) suggests that the peptides of the invention may interact with the same receptors as Neuropeptide Y (NPY) and PYY.

Glutamate is a potent excitatory neurotransmitter. Studies of NPY action in the hippocampus using brain slices in vitro suggest that NPY has a potent inhibitory action on the release of glutamate in the hippocampus through the NPY—Y2 receptors (Greber, S. et al (1994) Br. J. Pharmacol. 113(3):737-40). In other studies, it has been shown that NPY acts presynaptically in the hippocampal CA1 region to reduce excitatory input to the pyramidal neurones (Colmers, W. F. et al. (1987) J. Physiol. 383:285-299). It has been suggested that NPY inhibits excitatory synaptic transmission at the Schaffer collateral-CA1 synapse by acting directly at the terminal to reduce a Ca2+ influx (Colmers, W. F. et al. (1988) J. Neurosci. 8:3827-2837).

The neuropeptides of the invention are shown herein to increase or decrease synaptic transmission and are thus useful for modulating excitatory input into pyramidal neurons and lessen or increase glutamate release and subsequent excitatory effects. This discovery finds particular utility in modulating excitatory effects (presumably mediated through NPY and/or PYY receptors). As such, the peptides may be administered to a subject to modulate immune responses, gastrointestinal motility, pancreatic and adrenal function, eating activity, circadian rhythm, arousal, blood circulation and pressure, and cell proliferation in normal and malignant tissues.

The data indicate that the neuropeptides of the invention are useful for modulating biological effects involving NPY and PYY receptors. Therapies directed to modulation of NPY and PYY receptors are embraced by the invention. The peptides of the invention may be administered to subjects for the treatment of such disorders related to excitatory neurotransmission as neuropathic pain, epilepsy, stroke and brain damage in premature infants.

In a preferred embodiment, the peptide of SEQ ID NOs: 9 and/or 10 and/or 11 is administered to subjects for the treatment of such disorders related to excitatory neurotransmission as neuropathic pain, epilepsy, stroke and brain damage in premature infants.

The peptides of the invention may also be used in vitro to modulate excitatory neurotransmission.

7. Expression of Neuropeptides

As described above, expression of the polynucleotides encoding the neuropeptides of the invention may be effected by inserting the polynucleotides encoding the neuropeptides into an appropriate expression vector and introducing the polynucleotides into an appropriate cell type. Introduction of nucleic acid into cells may be by any method known in the art, including, but not limited to transfection (e.g., calcium phosphate precipitation, electroporation, gene gun “biolistic” techniques), transformation, or transduction.

The neuropeptides of the invention may also be expressed in vitro by using such a system as a rabbit reticulocyte lysate (RRL) system, a wheat germ agglutinin system, or any other in vitro expression system known in the art. Kits are available for in vitro translation systems and include RRL and wheat germ agglutinin kits produced by Promega. Methods of performing in vitro translation are provided by the manufacturer.

The expression of neuropeptides in Cos cells is disclosed below. The neuropeptides of the invention can also be expressed in other systems known in the art, such as bacteria, baculovirus or Drosophila Schneider-2 cells. A method for expressing proteins using a baculovirus is described in PCT/US96/18197 (incorporated herein by reference). Bacterial expression can be performed following techniques well known in the art, for example, Peränen J. et al. (1996) Anal. Biochem., 236:371-373 (incorporated by reference herein). An easy approach to use of Schneider-2 cells is offered by the Invitrogen Drosophila Expression system.

8. Methods of Treatment

The present invention provides for both prophylactic and therapeutic methods of treating a subject manifesting at least one sign of a motor disorder, neurodegenerative disease, epileptic syndrome, renal disorder, hair loss, and/or neuropathic pain. The invention also provides for the treatment of disorders associated with increased or decreased expression of neuropeptides derived from GDNF precursor or a homolog thereof. The compositions that are useful for treatment of these disorders may be therapeutic, or prophylactic. Diseases and disorders that are characterized by increased (relative to a subject not suffering from the disease or disorder) levels or biological activity may be treated with compositions that antagonize (i.e., reduce or inhibit) activity. Diseases and disorders that are characterized by decreased (relative to a subject not suffering from the disease or disorder) levels or biological activity may be treated with agonists (i.e., compounds that increase activity). The compositions that are suitable for use in the invention include, but are not limited to GDNF precursor-derived neuropeptides or analogs, derivatives, fragments or homologs thereof; anti-neuropeptide antibodies; nucleic acids encoding a GDNF precursor or homolog-related neuropeptide; modulators (i.e., inhibitors, agonists and antagonists, including additional peptide mimetic of the invention or antibodies specific to a peptide of the invention) that alter the interaction between a GDNF precursor or homolog-related neuropeptide.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a GDNF or homolog-derived neuropeptide, or analogs, derivatives, fragments or homologs thereof; an agonist that increases bioavailability; or agents that stimulate GDNF expression and/or proteolytic processing.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a GDNF or homolog-derived neuropeptide). In general, GDNF precursor-derived neuropeptides may be quantified in comparison to normal tissue. In addition, the amount of GDNF precursor or homologs thereof may be analyzed in relation to normal cells. RNA encoding GDNF precursor or homolog may also be used as an index of GDNF precursor expression which can be used to extrapolate the amount of GDNF precursor-derived neuropeptides (or homologs thereof) resulting from the precursors.

Methods that are well-known within the art to analyze nucleic acid and protein include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, reverse transcriptase polymerase chain reaction (rtPCR), etc.).

In one aspect, the invention provides a method for preventing a disease or condition associated with an aberrant GDNF precursor-derived neuropeptide (or homologs thereof) expression or activity, by administration of a composition that modulates expression of GDNF precursor-derived neuropeptides (or homologs thereof) or at least one neuropeptide activity. Compositions that modulate expression, proteolytic cleavage or activity of the GDNF precursor-derived neuropeptides of the invention may be used as treatments for conditions related to aberrant GDNF precursor-derived neuropeptide (or homolog thereof) expression or activity. Depending on the type of neuropeptide aberrancy, an agonist or antagonist can be used for treatment.

Another aspect of the invention pertains to methods of modulating the expression or activity of the neuropeptides of the invention by contacting a cell with a composition that modulates one or more of the activities of the neuropeptides of the invention. Such a composition may include a neuropeptide of the invention, or a fragment or analog thereof; an anti-neuropeptide antibody of the invention; a fusion protein containing a neuropeptide of the invention or fragment or analog thereof; at least a portion of an anti-neuropeptide antibody that specifically binds to a neuropeptide of the invention (which is optionally fused to another protein, or portion thereof); and/or a polynucleotide encoding a neuropeptide of the invention, or fragment, fusion protein, or analog thereof. The methods of the invention for treating a subject may be performed in vivo, ex vivo, or in vitro. The amounts of the therapeutic will vary according to the method and active ingredient used. Typically, the amount of neuropeptide, antibody or modulatory agent used will be an amount sufficient to achieve the desired effect without causing any untoward effects in the subject.

In some embodiments of the invention, peptides are administered to subjects to modulate the effects associated with PYY and/or NPY receptors. The neuropeptides of the invention may be used to up-regulate or down-regulate these effects. For example, and not by way of limitation, the neuropeptides of the invention may be used to compete with PYY and NPY to down-regulate the effects of NPY and/or PYY. Alternatively, the neuropeptides of the invention may be administered to stimulate an effect by interacting with NPY and/or PYY receptors. In preferred embodiments, the neuropeptides of the invention are administered to subjects as a means to cause vasoconstriction, cardiac stimulation, or inhibition of noradrenaline and/or renin release. In preferred embodiments of the invention, a peptide of the sequence shown in SEQ ID NO:9 and/or SEQ ID NO:10 and/or SEQ ID NO:11 is administered to a subject to modulate activity associated with NPY and/or PYY receptors.

Peptides of the invention may be administered alone, or n combination with at least one pharmaceutically acceptable carrier for the treatment of disorders associated with excitatory neurotransmission. Such disorders include, but are not limited to epilepsy, stroke and brain damage.

In preferred embodiments, the neuropeptides of the invention may be used to treat such diseases and disorders as renal disorder (e.g., the compositions of the invention are useful in promoting tubular regeneration following renal damage), motor diseases, neurodegenerative diseases, neuropathic pain and other neurological disorders such as epileptic syndromes. The neuropeptides of the invention may also be used to treat disorders of the enteric nervous system and may be used to treat skin disorders such as hair loss as a result of apoptosis-driven hair follicle involution.

The compositions of the invention may be formulated for enteric and parenteral use. Such formulations include at least one pharmaceutically acceptable carrier for delivering the neuropeptides of the invention or fragments thereof. The formulations may be made for delivery for any route of administration, including oral, nasal, rectal, topical, intramuscular, intradermal, interperitoneal, and subcutaneous routes. The compositions may also be formulated as inhalants.

The specific application may determine the optimal route of administration. As an example, but not by way of limitation, a composition of the invention containing at least one neuropeptide of the invention may be formulated as a topical composition when it is to be used for prevention of hair loss. As another non-limiting example, compositions for treating renal disease may be formulated as an oral composition or injectable composition. The person of ordinary skill in the art is acquainted with formulation strategies for optimal delivery of pharmaceuticals to particular tissues for optimal efficacy and ease of administration and would know how to modify the compositions for use. Such modifications are within the scope of the invention.

The invention is further described in the Examples as set forth below. Example 4 is a prophetic example. The examples are provided merely as illustrations of the embodiments of the invention and are not to be construed as limiting the scope of the invention which is set forth in the appended claims.

### EXAMPLES

### Example 1

### Identification of Vertebrate GDNF-Derived Neuropeptides

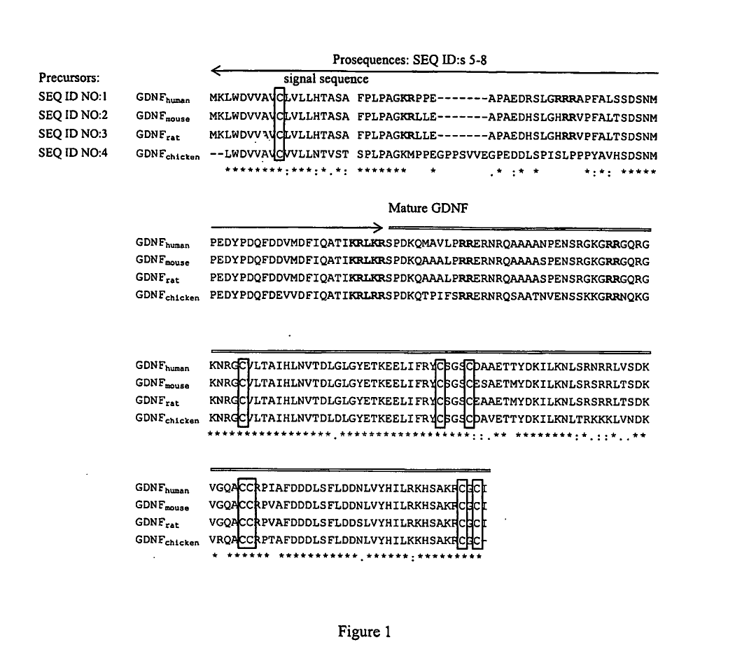

Analysis of the sequence of the human GDNF precursor revealed additional PC processing sites within the proregion (SEQ ID:s 5-8) and also in the amino-terminal half of the mature GDNF (SEQ ID:s 20-23) (FIG. 1). Processing at the indicated sites would result in formation of short (4-17 amino acids) peptides, and after successive action of carboxypeptidase E/H and peptidylglycine α-amidating monooxygenase PAM (Eipper B. et al. (1993) Prot. Sci. 2:489-497) three of them would yield amidated peptides (Table 1).

The cysteine knot of GDNF implicates strong structure-function relationship in GDNF as the cysteine-bonding pattern of the substrates affects the accessibility of putative processing sites. However, all the implicated peptide products originate either from the propeptide, which is separated from the folded GDNF precursor reaching the convertase responsible for GDNF maturation at late stage of the secretion pathway, or from the N-terminal part of the mature GDNF preceding the first cysteine. None of the implicated processing sites would thus be buried into the knot, and as the peptides do not contain Cys residues they can be released from the precursor by endopeptidase cleavage. Furthermore, formation of any of the suggested peptides would not affect the activity of mature GDNF (Baloh et al. (2000) J. Biol. Chem. 275(5), 3412-3420).

Other precursors of the GDNF family of neurotrophic factors (artemin, neurturin, persephin) do not contain similar good candidate sites for peptide production, and none of the putative weak processing sites would yield amidated peptides. Instead, human GDNF precursor contains several well-conserved substrate sites for furin/PC convertases and other enzymes required for release of neuropeptide-like products.

| | | | | |
|---|---|---|---|---|
| SEQ ID | | functional | | |
| NO: | name | name | sequence | species |

| | | | | |
|---|---|---|---|---|
| 9 | PEP1 | Neuron | FPLPA(a) | human, |
| | | Related | | mouse, rat |
| | | Peptide | | |
| | | NRP | | |
| 10 | hPEP2 | | PPEAPAEDRSL(a) | human |
| 11 | rPEP2 | Brain | LLEAPAEDHSL(a) | mouse, rat |
| | | Excitatory | | |
| | | Peptide | | |
| | | BEP | | |
| 12 | hPEP3 | | SPDKQMAVLP | human |
| 13 | rPEP3 | | SPDKQAAALP | mouse, rat |
| 14 | | | SPDKQTPIFS | chicken |
| 15 | hPEP4 | | ERNRQAAAANPENS | human |
| | | | RGK(a) | |
| 16 | rPEP4 | | ERNRQAAAASPENS | mouse, rat |
| | | | RGK(a) | |
| 17 | | | ERNRQSAATNVENS | chicken |
| | | | SKK(a) | |

Table 1 shows the sequences of the GDNF precursor-derived neuropeptides of human, mouse, rat and chicken. “a” indicates alpha amidation. The peptides shown are from human, mouse and rat (SEQ ID NO:9); human (SEQ ID NO:10); mouse and rat (SEQ ID NO:11); (SEQ ID NO:12); mouse and rat (SEQ ID NO:13); chicken (SEQ ID NO:14); human (SEQ ID NO:15); mouse and rat (SEQ ID NO:16); and chicken (SEQ ID NO:17). In the PEP-nomenclature of the peptides, rodent or human origin is indicated by the preceeding r- or h-, respectively, and the numbers refer to the order of the predicted peptides along the respective GDNF precursor from the N- to C-terminus.

### Example 2

### Sequence Analysis of the Human GDNF-Derived Neuropeptides

GDNF-derived neuropeptides were analyzed for similarity to other, known neuropeptides based on homology at the amino acid level.

Neuropeptides derived from the proregion of GDNF (SEQ ID NO:10 and SEQ ID NO:11) are highly homologous to a recently identified member of the neuropeptide Y (NPY) family. This new neuropeptide, the human peptide YY2 (hPYY2) (SEQ ID NO:18) (Couzens et al. (2000) Genomics 64 (3), 318-323), is the closest human equivalent of the long known bovine PYY2/seminaplasmin. An alignment of SEQ ID NO:10 and SEQ ID NO:11 with hPYY2 (SEQ ID NO:18) yields:

| | | | |
|---|---|---|---|
| SEQ ID NO:10 | | P--PEAPAEDRSL | (a) |
| | | * **** ** * | |
| SEQ ID NO:18 | hPYY2 | YPIKPEAPGEDAFL | (a) |
| | | *** ** * | |
| SEQ ID NO:11 | | LLEAPAEDHSL | (a) |

The amidation (a) of the hPYY2 is not certain, but identity between hPYY2 and SEQ ID NO:10 is 57-63%, depending on the region compared. The lower percentage relates to identity between SEQ ID NO:10 and the whole hPYY2. The higher percentage refers to identity between SEQ ID NO:10 and the eleven C-terminal amino acids of hPYY2.

The homologous region can also be found in other PYY and NPY-family peptides, but hPYY2 is the shortest member of this family and best homolog of SEQ ID NO:10 and SEQ ID NO:11.

NPY and its receptors (Yᵢ) are both expressed in hippocampal interneurones. In the rat hippocampus, application of NPY inhibits excitatory transmission in the CA1 area (Colmers et al. (1987) J. Physiol. 383:285-299; Colmers et al. (1988) J. Neurosci. 8:3827-3837. Activation of Yᵢ by NPY inhibits presynaptic voltage-operated calcium channels, resulting in a decrease in presynaptic calcium levels and concomitantly reduced transmitter release (Qian et al. (1997) J. Neurosci. 17:8169-8177). The homology between peptide YY (PYY) and the GDNF-derived peptides SEQ ID NO:10 and SEQ ID NO:11 suggests that these GDNF-derived peptides (SEQ ID NO:s 10 and 11) could act on the same receptors (Yᵢ) as NPY and PYY. This hypothesis gains strength from the experiments shown in Example 3 (below).

### Example 3

### Biological Effects of GDNF Precursor-Derived Neuropeptide

The human GDNF-derived neuropeptide that is closest to the N-terminus of the GDNF precursor is shown in SEQ ID NO:9. The biological effects of the SEQ ID NO:9 and SEQ ID NO:11 neuropeptides were studied using an acute rat hippocampal brain slice preparation. Wistar rats (P28-P44) were anesthetized with ketamine and medetomidine and transcardially perfused with ice-cold artificial cerebrospinal fluid (ACSF) containing (in mM) 124 NaCl, 5 KCl, 1.25 NaH₂PO₄, 26 NaHCO₃, 2 MgSO₄, 2 CaCl₂, and 10 glucose, continuously gassed with 5% CO₂-95% O₂. After decapitation the brains were exposed and 400-μm-thick transverse hippocampal slices cut with a vibratome while in high-sucrose solution containing (in mM) 230 sucrose, 3 KCl, 8 MgCl₂, 1.25 NaH₂PO₄, 26 NaHCO₃, 0.5 CaCl₂, and 25 glucose. The slices were allowed to recover in continuously gassed ACSF at 32° C. for 30 min and at room temperature for at least 30 min before use. While recorded, the slices were held at 32° C., continuously gassed with 5% CO₂-95% O₂ and perfused with ACSF at 3 ml/min. A bipolar stimulation electrode was placed in the stratum radiatum in the CA1 area. Extracellular responses were evoked by single stimuli (submaximal amplitude, duration 100 μs, frequency 0.05 Hz) and recorded from the stratum pyramidale using glass microelectrodes with a tip resistance of 2-6 MΩ when filled with extracellular solution containing (in mM) 150 NaCl, 5.4 KCl, 1 MgCl₂, 1.8 CaCl₂, and 5 Hepes, pH adjusted to 7.4 with NaOH. Results were analyzed using the WCP program (kindly provided by Dr. J. Dempster, University of Strathclyde, UK). Peptides (synthetic, unlabeled, with no additional residues but with C-terminal amidation) and N-ethylmaleimide (NEM; Sigma, St. Louis, Mo.), which selectively blocks signaling via pertussis-toxin sensitive G_{i/o}-proteins in the CNS (Morishita et al (1997) J. Neurosci. 17(3): 941-950) were delivered via bath perfusion at 3 ml/min. NEM has complex effects on baseline synaptic transmission and has been reported to cause either first an increase and then a decrease on pEPSPs (Morishita et al (1997) J. Neurosci. 17(3): 941-950) or only an increase (Tang and Lovinger (2000), J. Neurophysiol. 83(1): 60-69). In our experiments, NEM increased the stimulus-evoked responses in every slice recorded.

Extracellular population excitatory postsynaptic potentials (pEPSPs) and population spikes (pSPs) evoked by 0.05 Hz electrical stimulation of the Schaffer collateral fibres were recorded in hippocampus area CA1 of slices taken from P28-P35 rats. The pEPSPs and pSPs were significantly increased by 30-40% upon superfusion with 10-50 nM the SEQ ID NO:11 neuropeptide, suggesting that SEQ ID NO:11 neuropeptide enhances the excitatory synaptic input into primary cells in the CA1 area. SEQ ID NO:11 also increased presynaptic excitability, seen as an average increase of 34% in the presynaptic fiber volley. SEQ ID NO:9 attenuated the synaptic transmission, as demonstrated by the significant decrease of presynaptic fiber volley and pSPs (−24%). These effects were fully or almost fully reversed upon removal of the SEQ ID NO:9 or SEQ ID NO:11 neuropeptides from the superfusate.

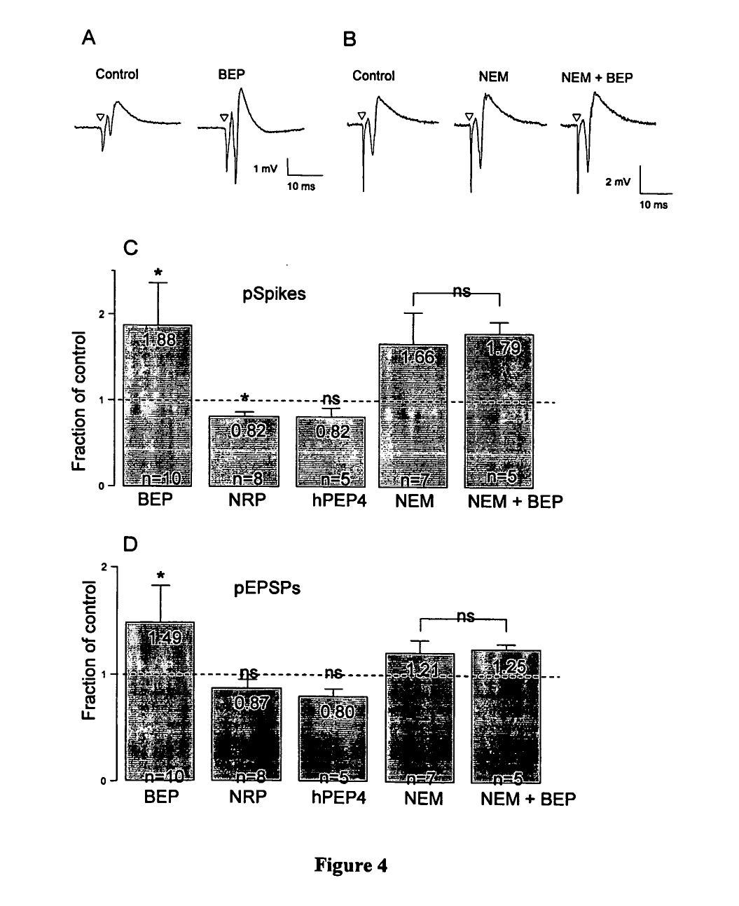

The observation that the effects of SEQ ID NO:s:9 and 11 on the hippocampal CA1 synapses resembles or opposes those observed for NPY and PYY suggests that the neuropeptides of the invention may act on the same receptors as NPY and/or PYY. The NPY receptors are coupled to G-proteins, preferentially to G_{i/o} (Wess, 1998). Application of SEQ ID NO: 11 in the presence of a G_{i/o}-blocking agent had no effect on excitability (FIG. 4 B, C, D), strongly suggesting that the excitatory action of SEQ ID NO:11 is also mediated via a presynaptic G_{i/o}-coupled receptor. The activity of SEQ ID NO:11 or SEQ ID NO:10 in human brain could not be tested. However, gdnf expression persists in the hippocampal formation until adulthood also in man (Serra et al. (2002), Brain Res. 928(1-2): 160-164).

### Example 4

### Detection of Secreted GDNF Precursor-Derived Peptides from Conditioned Media

Secreted GDNF precursor-derived peptides can be detected from conditioned media by transfecting cells (such as PC12, Neuro2A) with human pro-GDNF-encoding polynucleotides (e.g., mRNA transcribed in vitro from pSFV1-hgdnf construct (pSFV1: Gibco BRL)) and metabolically labelling the proteins of the transfected cells with a ¹⁴C-amino acid mixture. The medium from the cells can be collected and analyzed by sodium dodecyl sulfate polyacrylamide gel electrophoresis (SDS-PAGE) in Tris-Tricine buffers and visualized by standard procedures. Alternatively, secreted GDNF precursor-derived peptides can be detected from the growth media of cells, which are endogenously producing GDNF, such as the rat glial cell line C6 (ATCC CCL-107) (Suter-Crazzolara and Unsicker, (1996) Brain Res. Mol. Brain Res. 41(1-2): 175-182). Cells are grown to confluency in DMEM, 10% fetal calf serum before the media is changed to serum-free OPTIMEM I (Invitrogen, Carlsbad, Calif.) supplemented with 0.1% BSA. After two days the serum-free media is collected and Complete-Mini protease inhibitor cocktail tablets (Roche, Basel, Switzerland) added. Collected media can directly be subjected to mass spectrometric analysis with MALDI-TOF and ion-trap ESQUIRE, or be further purified with centrifugation through YM-10 and/or YM-3 columns (Millipore, Bedford, Mass.) and subsequent dialysis against ultra-pure water with MWCO 100 Da membrane (Spectrum Laboratories, Rancho Domingues, Calif.). If required, samples can be lyophilized and resuspended to 50× concentration in water.

### Example 5

### Binding of Iodinated Peptides to Neuronal Tissues

The binding of I¹²⁵-labeled peptides to embryonic and adult rodent tissues was studied in vitro. Chemically synthesized peptides containing an extra N-terminal tyrosine (Sigma-Genosys, Cambridge, UK) were labeled with lactoperoxidase (Trupp et al. (1995) J. Cell Biol. 130(1): 137-148). Specific activities obtained for each were 0.5-2.5×10¹⁴ cpm/mmol, except for SEQ ID NO:9 10¹³ cpm/mmol. From both E17 Wistar rat and E15 mouse embryos, kidneys, gut, pancreas and spinal cord were dissected. In addition, testes, ovaries, brain and adrenal glands of the embryonic mice were taken. The binding was performed and paraffin sections of embryonic tissues were processed as described (Partanen et al. (1987) Dev Biol 120(1): 186-197). Freshly cut adult rat brain slices of 1 mm were cut and incubated in 0.5-1 nmol/ml of ¹²⁵I-peptides, washed and processed for paraffin sections. Kodak (Rochester, N.Y.) NTB-2 emulsion was used for autoradiography and the samples were exposed from 3 to 12 weeks at +4° C. The sections were developed with Kodak D19 and stained with hematoxylin.

For immunohistochemical staining of tissues, paraffin sections were mounted with Immumount (Shandon, Pittsburgh, Pa.). Sections were stained with an anti-class III β-tubulin antibody (Tuj1; Covance Inc., Princeton, N.J.), which recognizes selectively neuronal cells. Staining was performed with Vectastain kit (Vector Laboratories, Burlingame, Calif.) according to manufacturer's instructions, except that the primary antibody (1:300) was incubated in +4° C. o/n.

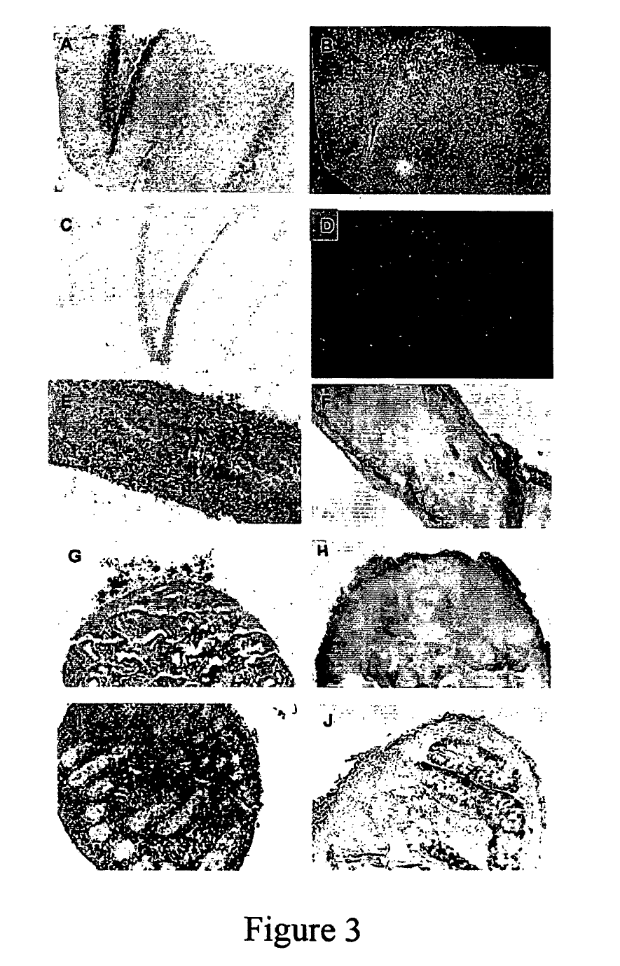

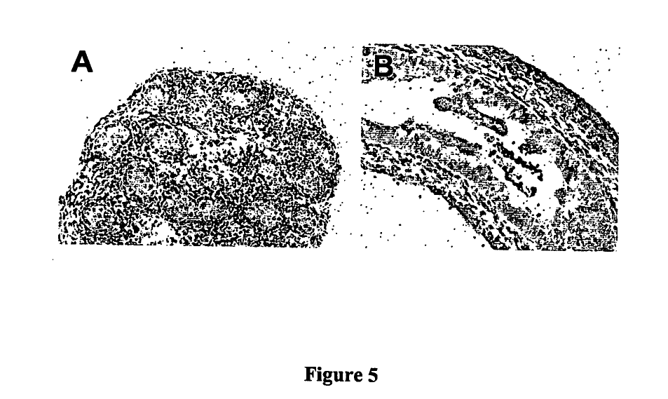

Only SEQ ID NO:s 9 and 11 resulted in specific binding. SEQ ID NO:11 (FIG. 3 A-B), but not SEQ ID NO:10 (FIG. 3 C-D), displayed extensive binding exclusively in adult rat brain (FIG. 3 A-D). Binding of SEQ ID NO:9 was restricted to embryonic mouse tissues outside the CNS. Distribution of the I¹²⁵-labelled SEQ ID NO:9 binding (FIG. 3 E, G) closely overlapped the neuronal β-tubulin staining (FIG. 3 F, H) both in the gut (FIG. 3 E, F) and the kidney capsule (FIG. 3 G, H) (FIG. 3 E-H). Although the autoradiographic detection of peptide binding in tissue does not allow localization of signal at the cellular level, it indicates that SEQ ID NO:9 was bound into neuronal or adjacent cells. The peptide-specificity of the binding patterns was demonstrated by the control peptide I¹²⁵-labelled SEQ ID NO:15 in embryonic mouse testis (FIG. 5A) and gut (FIG. 5B).

In testis, I¹²⁵-labelled SEQ ID NO:9 binding to the interstitium (I) was complementary both to the expression of GDNF and to the localization of neurons, detected by Tuj1 staining, within the seminiferous tubules (J).

Effective Concentrations of Peptides are Physiologically Relevant

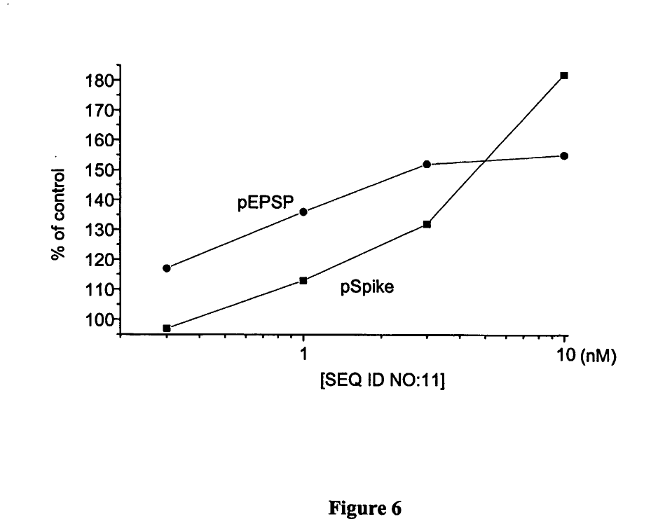

The nanomolar effective concentrations of SEQ ID NO:11 (FIG. 6) are remarkably lower than the 1 μM NPY and PYY₃₋₃₆ levels necessary for inhibition of synaptic transmission (Qian et al (1997), J. Neurosci. 17(21), 8169-8177). In fact, they are within the same range as the nanomolar GDNF concentrations required for promotion of ureteric branching (Sainio et al. (1997), Development 124(20), 4077-4087) or maintenance of primary neurons (U. Arumäe, personal communication). As SEQ ID NO:11 is synthesized in equimolar amounts to GDNF in vivo, the physiological concentration of SEQ ID NO:11 in vivo can reach the level required for synaptic excitation. This also suggests that the peptides of the invention may be more potent than NPY or any other known neuropeptide in modulating the excitatory response.

Various modification of the invention, in addition to those described herein, will be apparent to those skilled in the art from the foregoing description. Such modifications are also intended to fall within the scope of the appended claims.

1. An isolated polypeptide comprising a GDNF precursor or homolog thereof wherein said polypeptide comprises at least one neuropeptide activity.

2. The isolated polypeptide of claim 1 wherein said polypeptide is a native GDNF precursor isolated from cells.

3. The isolated polypeptide of claim 1 wherein said polypeptide is recombinantly produced.

4. The isolated polypeptide of claim 1 wherein said polypeptide is chemically synthesized.

5. The isolated polypeptide of claim 1 wherein said GDNF precursor comprises a vertebrate GDNF precursor fragment or homolog thereof.

6. The isolated polypeptide of claim 5 wherein said vertebrate is selected from the group consisting of bird, rodent, non-human primate and human.

7. The isolated polypeptide of claim 1 comprising an amino acid sequence selected from the group consisting of SEQ ID NO:5, SEQ ID NO:6, SEQ ID NO:7, and SEQ ID NO:8.

8. An isolated polypeptide comprising a fragment of a GDNF precursor or homolog thereof wherein said polypeptide comprises at least one neuropeptide activity.

9. The isolated polypeptide of claim 8 wherein said polypeptide is a proteolytically processed fragment.

10. The isolated polypeptide of claim 9 wherein said polypeptide is a native processed fragment of a GDNF precursor isolated from cells.

11. The isolated polypeptide of claim 8 wherein said polypeptide is recombinantly produced.

12. The isolated polypeptide of claim 8 wherein said polypeptide is chemically synthesized.

13. The isolated polypeptide of claim 8 wherein said GDNF precursor comprises a vertebrate GDNF precursor or homolog thereof.

14. The isolated polypeptide of claim 13 wherein said vertebrate is selected from the group consisting of bird, rodent, non-human primate and human.

15. The isolated polypeptide of claim 8 wherein said neuropeptide is α-amidated.

16. The isolated polypeptide of claim 8 comprising an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

17. The isolated polypeptide of claim 15 comprising an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

18. An isolated nucleic acid comprising a polynucleotide sequence encoding a GDNF precursor or homolog thereof, wherein said polynucleotide sequence encodes a polypeptide comprising at least one neuropeptide activity.

19. The isolated nucleic acid molecule of claim 18 wherein said GDNF precursor or homolog thereof comprises a vertebrate GDNF precursor.

20. The isolated nucleic acid of claim 18 wherein said GDNF precursor comprises an amino acid sequence selected from the group consisting of SEQ ID NO:5, SEQ ID NO:6, SEQ ID NO:7, and SEQ ID NO:8.

21. An isolated nucleic acid comprising a polynucleotide sequence encoding fragment of a GDNF precursor or homolog thereof, wherein said polynucleotide sequence encodes a polypeptide comprising at least one neuropeptide activity.

22. The isolated nucleic acid of claim 21 wherein said GDNF precursor or homolog thereof comprises a vertebrate GDNF precursor.

23. The isolated nucleic acid of claim 21 wherein said GDNF precursor comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

24. An antibody comprising at least a portion of an immunoglobulin that specifically binds to a polypeptide comprising a GDNF precursor or homolog thereof, wherein said polypeptide comprises at least one neuropeptide activity.

25. The antibody of claim 24 wherein said GDNF precursor comprises a vertebrate GDNF precursor.

26. The anti-neuropeptide antibody of claim 24 wherein said antibody is a monoclonal antibody.

27. The antibody of claim 24 wherein said antibody specifically binds to a polypeptide comprising an amino acid sequence selected from the group consisting of SEQ ID NO:5, SEQ ID NO:6, SEQ ID NO:7, and SEQ ID NO:8.

28. An antibody comprising at least a portion of an immunglobulin that specifically binds to a fragment of a GDNF precursor or homolog thereof, wherein said fragment comprises at least one neuropeptide activity.

29. The antibody of claim 28 wherein said GDNF precursor comprises a vertebrate GDNF precursor or homolog thereof.

30. The antibody of claim 28 wherein said antibody is a monoclonal antibody.

31. The antibody of claim 28 wherein said antibody specifically binds to a polypeptide comprising an amino acid sequence selected from the group consisting of SEQ ID NO:9, SEQ ID NO:10, SEQ ID NO:11, and SEQ ID NO:12, SEQ ID NO:13, SEQ ID NO:14, SEQ ID NO:15, SEQ ID NO:16, and SEQ ID NO:17.

32. A vector comprising a polynucleotide sequence encoding a polypeptide wherein said polypeptide comprises a GDNF precursor or homolog thereof and comprises at least one neuropeptide activity.

33. The vector of claim 32 wherein said vector is an expression vector that directs expression of said polynucleotide sequence encoding said polypeptide.

34. The vector of claim 32 wherein said polynucleotide sequence encodes a polypeptide comprising a polypeptide sequence selected from the group consisting of SEQ ID NO:5, SEQ ID NO:6, SEQ ID NO:7, and SEQ ID NO:8.

35. A vector comprising a polynucleotide sequence encoding a polypeptide wherein said polypeptide comprises a fragment of a GDNF precursor or homolog thereof and comprises at least one neuropeptide activity.

36. The vector of claim 35 wherein said vector is an expression vector that directs expression of said polynucleotide sequence encoding said neuropeptide.

37. The vector of claim 35 wherein said polynucleotide sequence encodes a polypeptide comprising a polypeptide sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

38. A transformed cell comprising a vector wherein said vector comprises a polynucleotide sequence encoding a polypeptide comprising a GDNF precursor or homolog thereof, wherein said polypeptide comprises at least one neuropeptide activity.

39. The transformed cell of claim 38 wherein said vector is an expression vector that directs expression of said polynucleotide sequence encoding said polypeptide.

40. The transformed cell of claim 38 wherein said polynucleotide sequence encodes a polypeptide comprising a polypeptide sequence selected from the group consisting of SEQ ID NO:5, SEQ ID NO:6, SEQ ID NO:7, and SEQ ID NO:8.

41. A transformed cell comprising a vector wherein said vector comprises a polynucleotide sequence encoding a polypeptide comprising a fragment of a GDNF precursor or homolog thereof, wherein said polypeptide comprises at least one neuropeptide activity.

42. The transformed cell of claim 41 wherein said vector is an expression vector that directs expression of said polynucleotide sequence encoding said polypeptide.

43. The transformed cell of claim 41 wherein said polynucleotide sequence encodes a polypeptide comprising a polypeptide sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

44. A method of producing a GDNF precursor comprising introducing an expression vector into a cell wherein said expression vector comprises a polynucleotide sequence encoding a GDNF precursor or homolog thereof, wherein said GDNF precursor comprises at least one neuropeptide activity.

45. A method of producing a fragment of a GDNF precursor comprising introducing an expression vector into a cell wherein said expression vector comprises a polynucleotide sequence encoding a fragment of a GDNF precursor or homolog thereof, wherein said fragment comprises at least one neuropeptide activity.

46. A composition comprising at least one polypeptide and at least one pharmaceutically acceptable carrier, wherein said polypeptide comprises a polypeptide sequence of a GDNF precursor or homolog thereof, and wherein said polypeptide comprises at least one neuropeptide activity.

47. The composition of claim 46 wherein said polypeptide comprises an amino acid sequence selected from the group consisting of SEQ ID NO:5, SEQ ID NO:6, SEQ ID NO:7, and SEQ ID NO:8.

48. A composition comprising at least one polypeptide and at least one pharmaceutically acceptable carrier, wherein said polypeptide comprises a fragment of a GDNF precursor or homolog thereof, and wherein said polypeptide comprises at least one neuropeptide activity.

49. The composition of claim 48 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

50. A composition comprising an antibody, or portion thereof and at least one pharmaceutically acceptable carrier, wherein said antibody or portion thereof specifically binds to a polypeptide having an amino acid sequence of a GDNF precursor or homolog thereof.

51. The composition of claim 50 wherein said polypeptide comprises an amino acid sequence selected from the group consisting of SEQ ID NO:5, SEQ ID NO:6, SEQ ID NO:7, and SEQ ID NO:8.

52. A composition comprising an antibody or portion thereof and at least one pharmaceutically acceptable carrier, wherein said antibody of portion thereof specifically binds to a polypeptide having an amino acid sequence of a fragment of a GDNF precursor or homolog thereof.

53. The composition of claim 52 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

54. A method of modulating at least one activity of a neuron comprising administering to said neuron a composition comprising a polypeptide wherein said polypeptide comprises an amino acid sequence of a GDNF precursor or homolog thereof, wherein said polypeptide comprises at least one neuropeptide activity.

55. The method of claim 54 wherein said polypeptide is selected from the group consisting of SEQ ID NO:5, SEQ ID NO:6, SEQ ID NO:7, and SEQ ID NO:8.

56. A method of modulating at least one activity of a neuron comprising contacting with said neuron a composition comprising a polypeptide wherein said polypeptide comprises an amino acid sequence of a fragment of a GDNF precursor or homolog thereof, wherein said polypeptide comprises at least one neuropeptide activity.

57. The method of claim 56 wherein said polypeptide is selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

58. (canceled)

59. (canceled)

60. A method of modulating cRet activity comprising administering to a patient in need thereof a composition comprising at least one polypeptide having an amino acid sequence of a GDNF precursor or homolog thereof, wherein said polypeptide comprises at least one neuropeptide activity.

61. A method of modulating cRet activity comprising administering to a patient in need thereof a composition comprising at least one polypeptide having an amino acid sequence of a fragment of a GDNF precursor or homolog thereof, wherein said polypeptide comprises at least one neuropeptide activity.

62. The method of claim 60 or 61 wherein said cRet activity is down-modulated or up-regulated.

63. A method of modulating NPY—Yᵢ activity comprising administering to a patient in need thereof a composition comprising at least one polypeptide having an amino acid sequence of a GDNF precursor or homolog thereof, wherein said polypeptide comprises at least one neuropeptide activity.

64. A method of modulating NPY—Yᵢ activity comprising administering to a patient in need thereof a composition comprising at least one polypeptide having an amino acid sequence of a fragment of a GDNF precursor or homolog thereof, wherein said polypeptide comprises at least one neuropeptide activity.

65. The method of claim 63 or 64 wherein said NPY—Yᵢ activity is down-modulated or up-regulated.

66. A method of treating a motor disease comprising administering to a patient manifesting at least one sign of a motor disease a composition comprising at least one polypeptide having an amino acid sequence of a GDNF precursor or homolog thereof, wherein said polypeptide comprises at least one neuropeptide activity.

67. The method of claim 66 wherein said polypeptide comprises an amino acid sequence selected from the group consisting of SEQ ID NO:5, SEQ ID NO:6, SEQ ID NO:7 and SEQ ID NO:8.

68. A method of treating a motor disease comprising administering to a patient manifesting at least one sign of a motor disease a composition comprising at least one polypeptide having an amino acid sequence of a fragment of a GDNF precursor or homolog thereof, wherein said polypeptide comprises at least one neuropeptide activity.

69. The method of claim 68 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

70. The method of claim 66 or 68 wherein said motor disease is selected from the group consisting of chronic inflammatory demyelinating polyneuropathy, Parkinson's Disease, demyelinating Guillain-Barre syndrome, amyotrophic lateral sclerosis, and motor neuropathy.

71. A method of treating neuropathic pain comprising administering to a patient manifesting at least one sign of a neuropathic pain a composition comprising at least one polypeptide having an amino acid sequence of a GDNF precursor or homolog thereof, wherein said polypeptide comprises at least one neuropeptide activity.

72. The method of claim 71 wherein said polypeptide comprises an amino acid sequence selected from the group consisting of SEQ ID NO:5, SEQ ID NO:6, SEQ ID NO:7 and SEQ ID NO:8.

73. A method of treating neuropathic pain comprising administering to a patient manifesting at least one sign of a neuropathic pain a composition comprising at least one polypeptide having an amino acid sequence of a fragment of a GDNF precursor or homolog thereof, wherein said polypeptide comprises at least one neuropeptide activity.

74. The method of claim 73 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

75. A method of treating a motor disease comprising administering to a patient manifesting at least one sign of a motor disease a composition comprising an antibody or portion thereof that specifically binds to polypeptide having an amino acid sequence of a GDNF precursor or homolog thereof, wherein said polypeptide comprises at least one neuropeptide activity.

76. The method of claim 75 wherein said polypeptide comprises an amino acid sequence selected from the group consisting of SEQ ID NO:5, SEQ ID NO:6, SEQ ID NO:7, and SEQ ID NO:8.

77. A method of treating a motor disease comprising administering to a patient manifesting at least one sign of a motor disease a composition comprising an antibody or a portion thereof specifically binds to polypeptide having an amino acid sequence of a fragment of a GDNF precursor or homolog thereof, wherein said polypeptide comprises at least one neuropeptide activity.

78. The method of claim 77 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

79. The method of claim 75 or 77 wherein said motor disease is selected from the group consisting of chronic inflammatory demyelinating polyneuropathy, Parkinson's Disease, demyelinating Guillain-Barre syndrome, amyotrophic lateral sclerosis, and motor neuropathy.

80. A method of treating neuropathic pain comprising administering to a patient manifesting at least one sign of a neuropathic pain a composition comprising an antibody or a portion thereof that specifically binds to a polypeptide sequence of a GDNF precursor or homolog thereof, wherein said polypeptide comprises at least one neuropeptide activity.

81. The method of claim 80 wherein said polypeptide comprises an amino acid sequence selected from the group consisting of SEQ ID NO:5, SEQ ID NO:6, SEQ ID NO:7, and SEQ ID NO:8.

82. A method of treating neuropathic pain comprising administering to a patient manifesting at least one sign of a neuropathic pain a composition comprising an antibody or a portion thereof that specifically binds to a polypeptide sequence of a fragment of a GDNF precursor or homolog thereof, wherein said polypeptide comprises at least one neuropeptide activity.

83. The method of claim 82 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

84. (canceled)

85. (canceled)

86. (canceled)

87. (canceled)

88. A method of treating a testicular disorder comprising administering to a patient manifesting at least one sign of reduced spermatogenesis a composition comprising at least one polypeptide having an amino acid sequence of a fragment of a GDNF precursor or homolog thereof, wherein said polypeptide comprises at least one neuropeptide activity.

89. The method of claim 88 wherein said polypeptide comprises an amino acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

90. A method of modulating excitatory neurotransmission comprising administering to a subject an effective amount of a polypeptide comprising an amino acid sequence of a fragment of a GDNF precursor or homolog thereof, wherein said polypeptide comprises at least one neuropeptide activity.

91. The method of claim 90 wherein said polypeptide is selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

92. The method of claim 90 wherein said polypeptide is selected from the group consisting of SEQ ID NO:9, SEQ ID NO:10, and mixtures thereof.

93. A method of treating a disorder associated with excitatory neurotransmission comprising administering to a subject in need of treatment an effective amount of at least one polypeptide comprising a fragment of a GDNF precursor or homolog thereof, wherein said polypeptide comprises at least one neuropeptide activity.

94. The method of claim 93 wherein said disorder is selected from the group consisting of epilepsy, stroke, neurogenerative disorders, infantile brain damage, and neuropathic pain.
